(19) United States
(12) Patent Application Publication
(10) Pub. No.: US 2025/0127753 A1
(43) Pub. Date: Apr. 24, 2025

(54) PHARMACEUTICAL APPLICATION OF DIMYRICETIN-YL-DISELENIDE IN TUMORS

(71) Applicant: Shanghai Spark Pharmaceutical Co., Ltd., Shanghai (CN)

(72) Inventors: Kunyuan SONG, Shanghai (CN); Weiwei CHEN, Shanghai (CN)

(21) Appl. No.: 18/910,390

(22) Filed: Oct. 9, 2024

(30) Foreign Application Priority Data
Oct. 23, 2023 (CN) 202311379825.4

(51) Int. Cl. A61K 31/352; A61P 35/00

(52) U.S. Cl. CPC A61K 31/352; A61P 35/00

(57) ABSTRACT

This application discloses the medicinal application of dimyricetin-yl-diselenide in tumors. Experiments have confirmed that dimyricetin-yl-diselenide has the potential for tumor treatment.

### TECHNICAL FIELD

This application relates to the field of medical technology, in particular to the pharmaceutical application of a dimyricetin-yl-diselenide in tumors.

### BACKGROUND OF RELATED ARTS

Selenium is an essential trace element for human beings. The Chinese Nutrition Society has also listed selenium as one of the 15 essential nutrients for the human body. Numerous clinical trials both domestically and internationally have shown that selenium deficiency in the human body can lead to functional disorders of certain vital organs, resulting in the occurrence of many serious diseases. Studies indicate that individuals with low selenium levels or deficiency can prevent the occurrence of tumors, liver diseases, and other conditions by supplementing with an appropriate amount of selenium. Moreover, it can enhance the immune system, maintain the normal functions of essential organs such as the heart, liver, lungs, and stomach, and prevent the occurrence of geriatric cardiovascular and cerebrovascular diseases.

Myricetin is a flavonoid compound widely found in the bark and leaves of waxberry trees. It possesses various pharmacological activities, including anti-inflammatory, anti-tumor, anti-mutagenic, anti-caries, antioxidant properties, and the ability to eliminate free radicals in the body.

Diselenides exhibit significant antioxidant effects, mimicking the activity of glutathione peroxidase (GSH-PX). Some diselenides also demonstrate anti-tumor, antibacterial, disinfectant, and sterilization properties.

Dimyricetin-yl-diselenide (DMS), with a molecular formula of C30H18O16Se2 and a molecular weight of 792.37, is a yellowish powder. It is a novel compound discovered by our company.

### SUMMARY

The present application adopts the following technical solution:

The application of dimyricetin-yl-diselenide in the preparation of a medicament for treating tumors.

A method for inhibiting the growth of tumor cells in vitro in a non-therapeutic manner, comprising the steps of: adding dimyricetin-yl-diselenide to a tumor cell culture system, thereby inhibiting the growth of tumor cells.

The beneficial effects of the present application are as follows:

Dimyricetin-yl-diselenide has been experimentally proven to have the potential for tumor treatment.

### BRIEF DESCRIPTION OF THE DRAWINGS

The drawings described herein are used to provide a further understanding of the present application and constitute a part of the present application. The illustrative embodiments of the present application and the description thereof are used to explain the present application and do not constitute undue limitation to the present application. In the attached drawings:

FIGS. 1-7 show the results of in vitro cellular experiments, which include the IC50 values of Cpds for T24, MBT-2, HCC1954, EMT-6, HCT 116, CT-26.WT, MKN45, MFC, 786-O, Renca, Hep G2, H22, HCC827, LL/2 (LLC1), A375, B16-F10, PANC-1, Panc 02, DU145, RM-1, and SK-OV-3 cells.

FIG. 8 presents the results of animal experiments.

FIG. 9 also depicts the results of animal experiments.

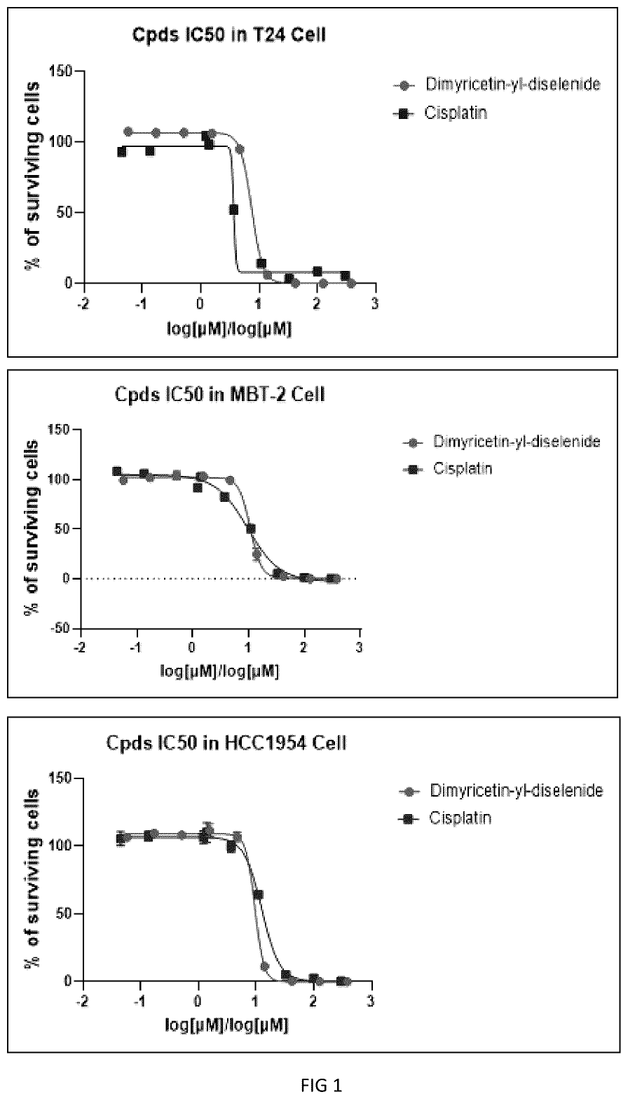

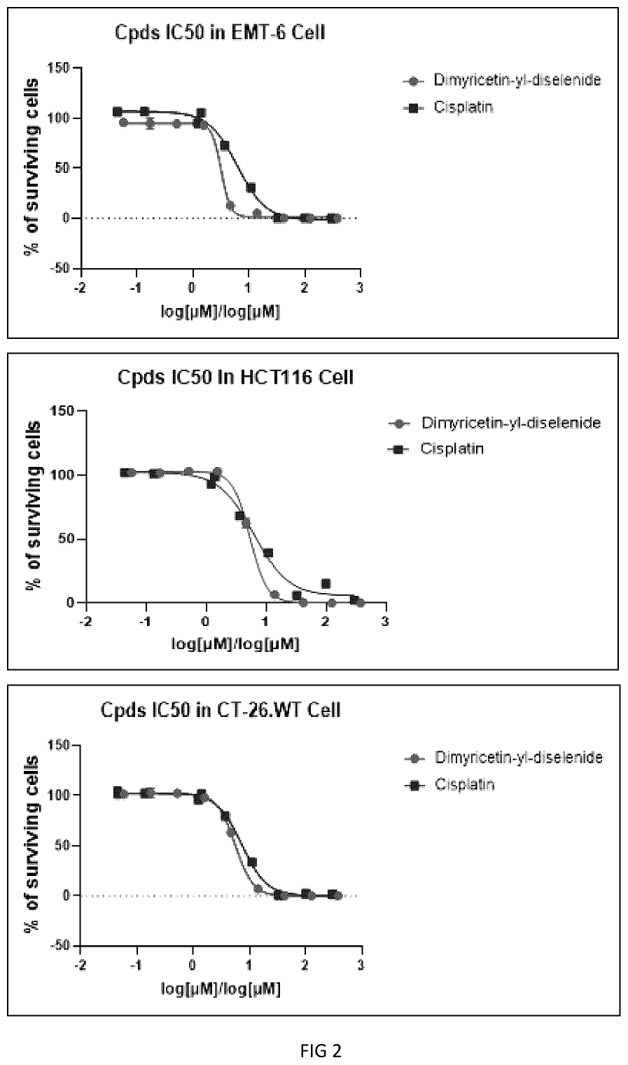

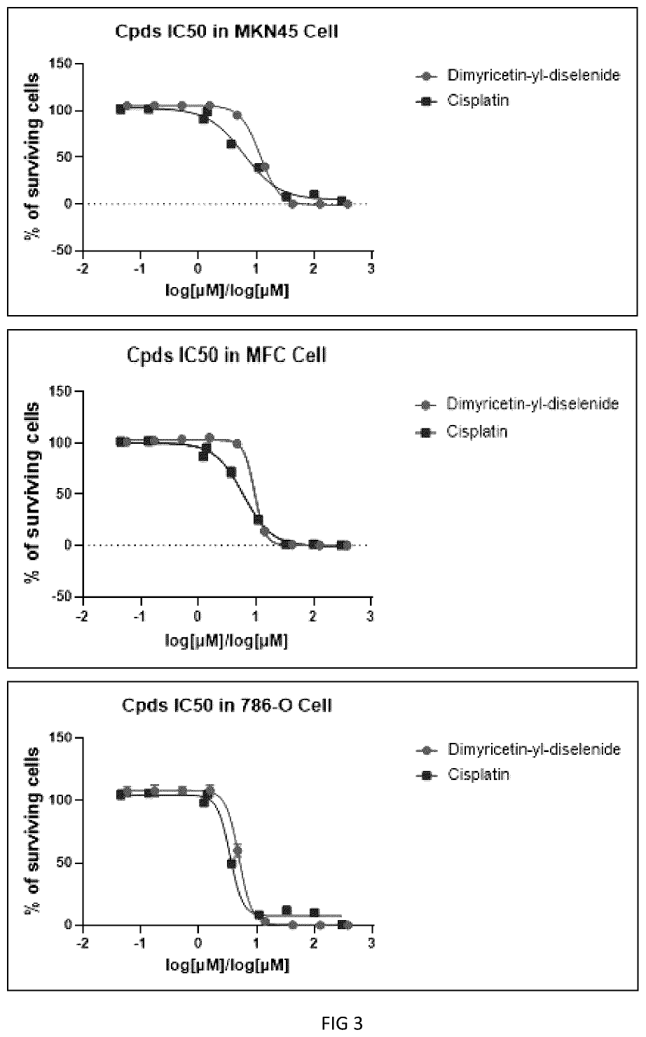

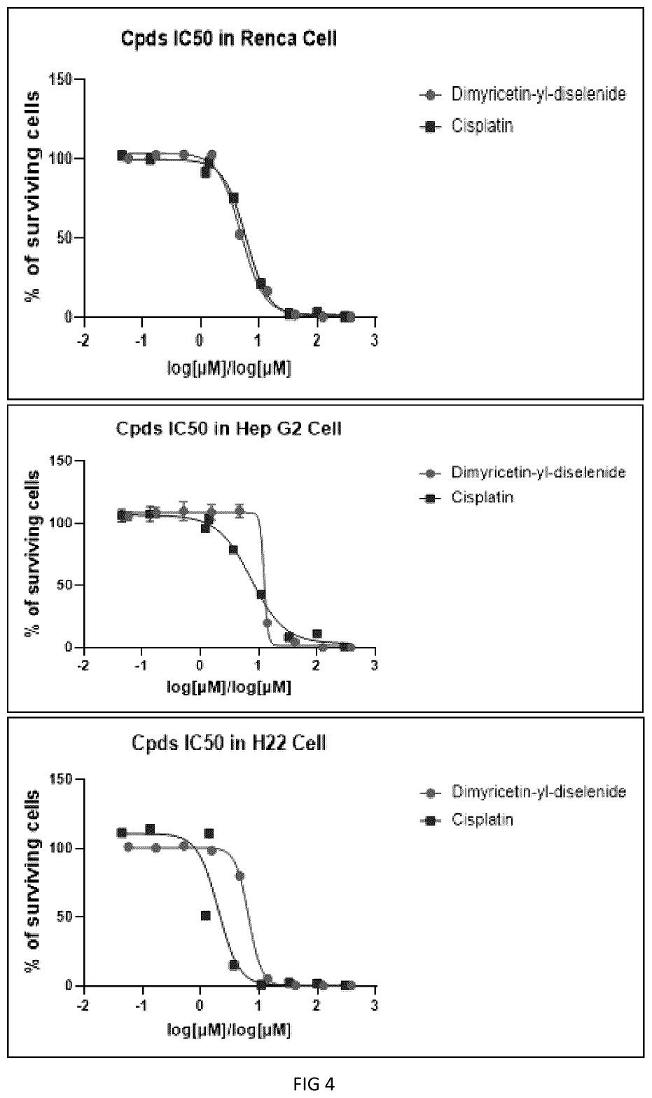

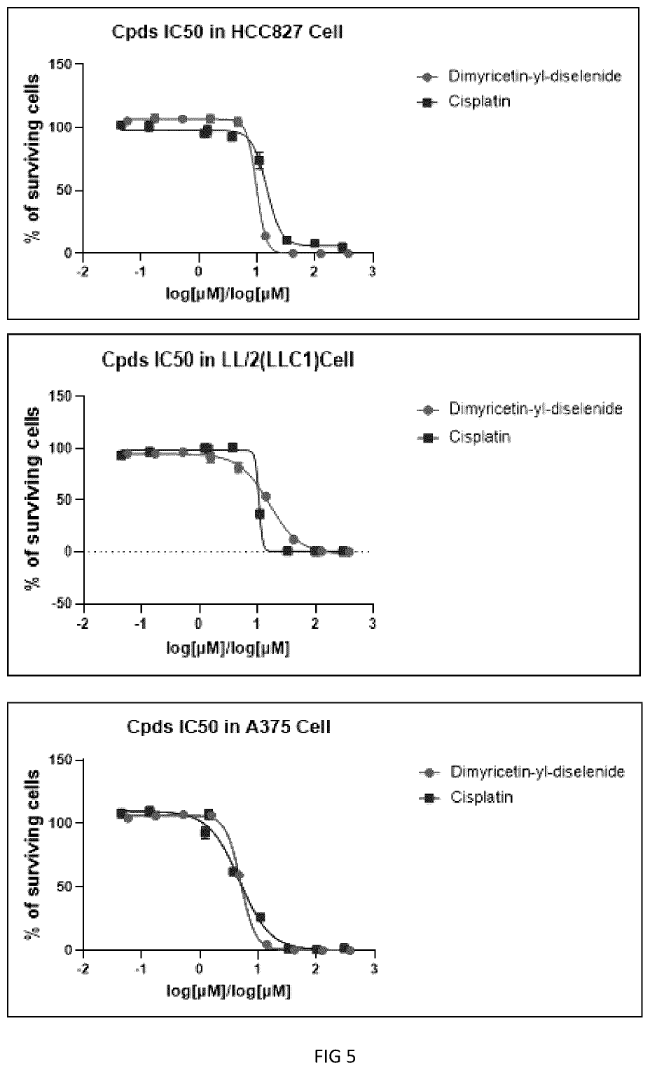

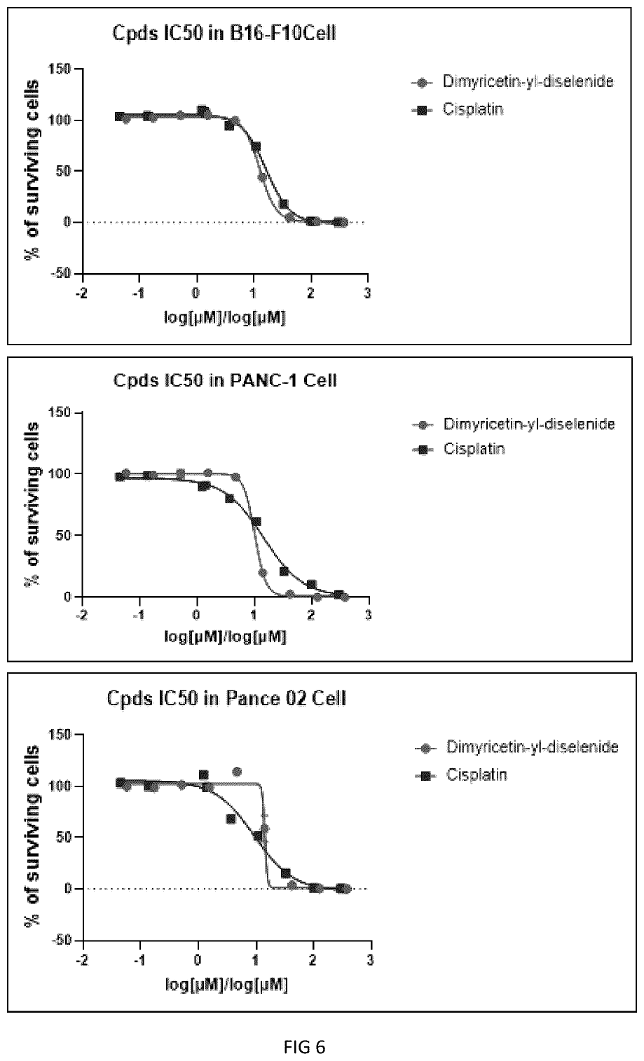

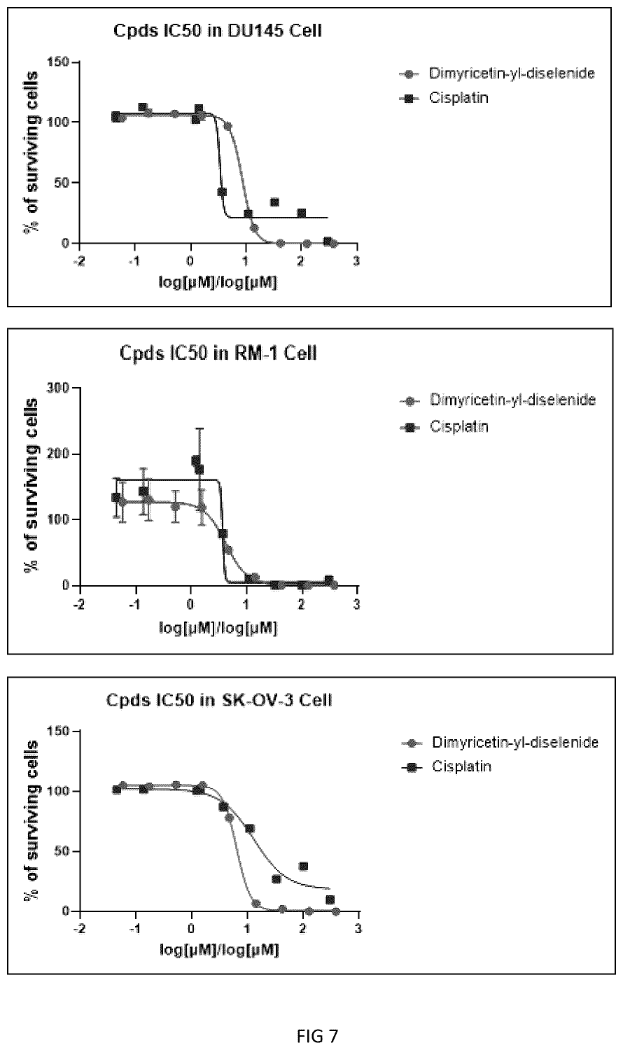

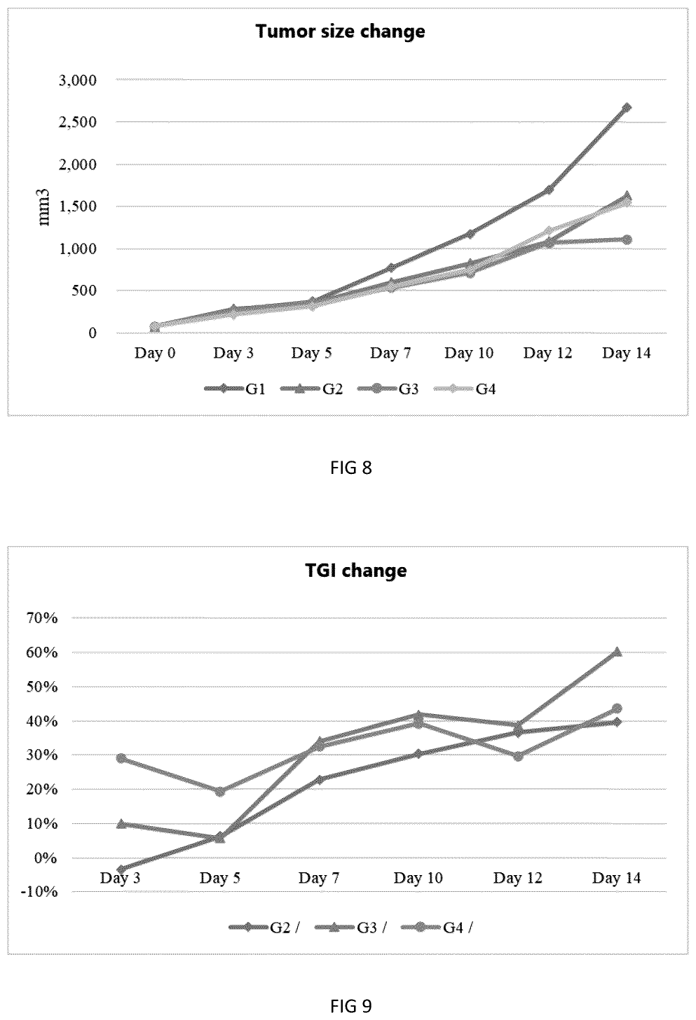

### DETAILED DESCRIPTIONS OF EMBODIMENTS

In order to make the purpose, technical scheme and advantages of this application clearer, the technical scheme of this application will be described clearly and completely in combination with the specific embodiments of this application and the corresponding drawings. Obviously, the described embodiments are only part of the embodiments of this application, rather than all embodiments. Based on the embodiments in this application, all other embodiments obtained by ordinary technicians in the field without creative work belong to the protection scope of this application.

### Example 1: In Vitro Cell Experiment

### 1) Cell Strain

| | | Cell | | |
|---|---|---|---|---|
| No. | Chinese Name | Name | Species | Culture Medium |
| 1 | Human bladder | T24 | Human | McCoy's 5a + 10% FBS |
| | transitional cell | | | |
| | carcinoma | | | |
| 2 | Mouse bladder | MBT-2 | Mouse | RPMI1640 + 10% FBS |
| | transitional cell | | | |
| | carcinoma | | | |
| 3 | Human breast cancer | HCC1954 | Human | RPMI1640 + 10% FBS |
| | cells | | | |
| 4 | Mouse breast cancer | EMT-6 | Mouse | RPMI1640 + 10% FBS |
| | cells | | | |
| 5 | Human colorectal | HCT 116 | Human | 90% McCoy's 5A + 10% h.i. FBS + |
| | adenocarcinoma | | | 2 mM L-glutamine (alternatively |
| | cells | | | Dulbecco's MEM with high glucose) |
| 6 | Mouse colorectal | CT-26.WT | Mouse | RPMI1640 + 10% FBS |
| | cancer cells | | | |
| 7 | Human gastric | MKN45 | Human | RPMI1640 + 20% FBS |
| | cancer cells | | | |
| 8 | gastric cancer cells | MFC | Mouse | RPMI1640 + 10% FBS |
| | of mice | | | |
| 9 | human renal | 786-O | Human | RPMI1640 + 10% FBS |
| | carcinoma cell | | | |
| 10 | mouse renal | Renca | Mouse | RPMI-1640 + 10% FBS + 1% P/S + |
| | adenocarcinoma | | | 0.1 mM NEAA + 1 mM Sodium |
| | cells | | | Pyruvate) + 2 mM L-glutamine |
| 11 | human | Hep G2 | Human | MEM + 10% FBS + 1% NEAA + 1 mM |
| | hepatocellular | | | Sodium Pyruvate (NaP) |
| | carcinoma cell | | | |
| 12 | mouse hepatocellular | H22 | Mouse | RPMI1640 + 10% FBS |
| | carcinoma cell line | | | |
| 13 | Human lung cancer | HCC827 | Human | RPMI1640 + 10% FBS |
| | cells | | | |
| | (adenocarcinoma) | | | |
| 14 | mouse lung cancer | LL/2 | Mouse | TBD |
| | cells | (LLC1) | | |
| 15 | human malignant | A375 | Human | DMEM + 10% FBS |
| | melanoma cells | | | |
| 16 | mouse melanoma | B16-F10 | Mouse | RPMI-1640 + 10% FBS + 1% P/S |
| | cells | | | |
| 17 | Human pancreatic | PANC-1 | Human | DMEM + 10% FBS |
| | cancer tumor cells | | | |
| 18 | mouse pancreatic | Panc 02 | Mouse | DMEM + 10% FBS |
| | cancer cells | | | |
| 19 | human prostate | DU145 | Human | MEM + 10% FBS + 1% NEAA |
| | cancer cell | | | |
| 20 | mouse prostate | RM-1 | Mouse | DMEM + 10% FBS + 1% P/S |
| | cancer cells | | | |
| 21 | human ovarian | SK-OV-3 | Human | McCoy's 5a + 10% FBS |
| | cancer cells | | | |

### 2) Experimental Materials and Reagents

DMEM, Product Code: 10-013-CV, Batch Number: 01222005;

FBS, Product Code: 10099-141C, Batch Number: 2544621CP;

96-well Flat Bottom Plate Corning®, Product Code: 062096, Batch Number: 2022010601;

Cell Titer-Glo (CTG) Novozymes, Product Code: DD1101-03, Batch Number: 7E522L1;

### 3) Equipment

Centrifuge, ThermoFisher, Model: ThermoFisher legend MACH 1.6R;

Inverted Microscope, Nikon, Model: TS100;

Biological Safety Cabinet, ThermoFisher, Model: Thermo MSC-Advantage;

CO2 Incubator, ThermoFisher, Model: Thermo HERA cell 150;

### 4) Test Drug

| | | | Quality | | Storage | molecular | |
|---|---|---|---|---|---|---|---|
| Compound | Cat. # | Lot. # | (mg) | Status | conditions | weight | solvent |
| Cisplatin | H20073652 | AA1A9011B | / | Liquid | 4° C. | 298.03 | / |
| Dimyricetin-yl- | | 210701R | 500 | Yellow | 4° C., | 792.37 | DMSO |
| diselenide | | | | powder | refrigerate, | | |
| | | | | | seal | | |

### 5) Cell Inoculation

Collect cells in the exponential growth phase and count viable cells separately using a cell counter. Adjust the cell suspension to an appropriate concentration with the corresponding culture medium. Add 90 μL of the cell suspension to each well of a 96-well cell culture plate, with 3 replicate wells containing 5000 cells/well. Place the plate in an incubator at 37° C. with 5% CO2 overnight.

On the second day, add 10 μL of dimyricetin diselenide and cisplatin to the corresponding wells. The highest final concentration of dimyricetin diselenide is 300 μg/ml (i.e., 379 μM), and the highest final concentration of cisplatin is 300 μM. A total of 9 concentrations are prepared with a 3-fold dilution.

After 72 hours, detect cell viability using the CTG assay.

### 6) Data Analysis

Using GraphPad Prism 8.0 software, an S-shaped dose-survival curve was plotted with log (inhibitor) as the x-axis and response as the y-axis, adopting the Variable slope (four parameters) model to calculate the IC50 value.

### 7) Test Results

| | | Dimyricetin-yl-diselenide | Cisplatin |
|---|---|---|---|
| No. | Cell | IC50 (μM) | IC50 (μM) |
| 1 | T24 | 7.69 | 3.71 |
| 2 | MBT-2 | 10.81 | 9.91 |
| 3 | HCC1954 | 9.86 | 13.21 |
| 4 | EMT-6 | 3.16 | 6.58 |
| 5 | HCT116 | 5.52 | 7.01 |
| 6 | CT-26.WT | 5.65 | 7.48 |
| 7 | MKN45 | 11.99 | 6.7 |
| 8 | MFC | 9.55 | 5.96 |
| 9 | 786-O | 5.14 | 3.68 |
| 10 | Renca | 5.17 | 6.03 |
| 11 | Hep G2 | 12.62 | 8.49 |
| 12 | H22 | 6.64 | 2.17 |
| 13 | HCC827 | 10 | 15.1 |
| 14 | LL/2(LLC1) | 14.59 | 10.57 |
| 15 | A375 | 5.2 | 5.36 |
| 16 | B16-F10 | 13.06 | 16.94 |
| 17 | PANC-1 | 10.28 | 13.8 |
| 18 | Pance 02 | 14.29 | 9.86 |
| 19 | DU145 | 8.79 | 3.59 |
| 20 | RM-1 | 5.12 | 3.83 |
| 21 | SK-OV-3 | 6.52 | 18.03 |

The results showed that dimyricetin-yl-diselenide had a significant inhibitory effect on tumor cells in vitro, and its inhibitory effect on some cells even exceeded that of positive drugs.

### Example 2: Study on the Pharmacodynamics of Subcutaneous Transplanted Tumor In Vivo

### 1) Tested Substance

| | |
|---|---|
| Positive control: | Cisplatin for Injection |
| Supplier: | PengLi Biology |
| manufacturer: | Qilu Pharmaceutical (Hainan) Co., Ltd. |
| Trait: | Freeze-dried agent |
| Batch No.: | FA2A1024B |
| Specification: | 20 mg/ [symbol] |
| Storage | keep away from light, Keep airtight |
| conditions: | |
| Expiration date: | Nov. 3, 2024 |
| Target | Dimyricetin-yl-diselenide |
| compound: | |
| Test substance | 210701R |
| batch number: | |
| Supplier: | Shanghai Spark Pharmaceutical Co., Ltd. |
| Effectiveness: | The test substance |
| Trait: | yellow powder |
| Storage | seal, refrigeration |
| conditions: | |

### 2) Reagent

| Reagent | Company | Product Code |
|---|---|---|
| RPMI1640 culture medium | Corning cellgro | 10-040-CV |
| Fetal bovine serum (FBS) | GIBCO | 10270-106 |
| Penicillostreptomycin (P.S.) | GIBCO | 15140-122 |
| trypsin-ethylenediaminetetraacetic acid | GIBCO | 25200-072 |
| PBS | Corning cellgro | 21-040-CVR |

### 3) Instrument

| Instrument | Company | Product Code |
|---|---|---|
| centrifuge | ThermoFisher | Thermo Fisher legend MACH 1.6/ |
| inverted microscope | Nikon | TS100 |
| Biosafety cabinet (cell culture) | ThermoFisher | 1379 |
| CO2 incubator | ThermoFisher | Thermo HERA cell 150 |
| water bath pot | Shanghai Jinghong | DK-8B |
| liquid nitrogen tank | ThermoFisher | Thermo 7403 |
| Pipette (electric) (cell culture) | Capp | PA-100 |
| Pipette (1 ml) (cell culture) | Eppendorf | Research plus |
| Pipette (200 μl) (cell culture) | Eppendorf | Research plus |
| Pipette (10 μl) (cell culture) | Eppendorf | Research plus |
| anesthesia machine | MATRX | MATRX VIP3000 |
| Biological safety cabinet (cell | ThermoFisher | Thermo MSC- Advantage |
| inoculation) | | |
| biosafety cabinet (for dispensing) | ThermoFisher | Thermo MSC- Advantage |
| scale (animal weight) | American Denver | DENVER INSTRUMENT TP-602 |
| Vernier caliper | Mitutoyo Japan | Mitutoyo CD-15cX |

### 4) Experimental Procedures

### a) Animal Reception, Evaluation, and Housing

Animal species and strain: BALB/c mice
Drug administration history: None
Gender and age: Female, 6-7 weeks old
Breeder/supplier: Shanghai Jihui Laboratory Animal Breeding Co., Ltd. or other qualified suppliers
Experimental facility: Pengli Biology Animal Facility
Acclimation period: 3-7 days
Number of animals: 42
Housing: SPF (Specific Pathogen Free) area
Indoor temperature: 20-26° C.
Indoor relative humidity: 40-70%
Lighting: Fluorescent lighting, 12 hours on (08:00-20:00) and 12 hours off
Animal housing: 2-6 mice per cage (same treatment group)
Food: Ad libitum access to irradiated feed (supplied by Jiangsu Xie Tong Pharmaceutical Bioengineering Co., Ltd., China)
Water: Ad libitum access to drinking water (tap water treated by reverse osmosis or autoclaving and acidified)

BALB/c mice were purchased from Shanghai Jihui Laboratory Animal Breeding Co., Ltd. for the H22 efficacy experiment. The animals were free of specific pathogens and arrived at Pengli Biology Animal Facility at approximately 5-6 weeks of age. The proposed animal handling procedures for the experimental protocol will be reviewed and approved by the IACUC (Institutional Animal Care and Use Committee) of Pengli Biology before the start of the experiment.

Upon arrival at Pengli Biology, animals were transferred from the transport containers to cages by the animal facility staff, and each animal was examined for any abnormalities in appearance, limbs, orifices, and behavior both at rest and in motion. An acclimation period of 3-7 days was provided.

### b) Cell Culture and Inoculation

The H22 cell line used in this experiment was cultured in RPMI-1640 medium supplemented with 10% FBS in a CO2 incubator at 37° C. Prior to continuous culturing for ten passages, approximately 3×106 H22 cells were suspended in PBS and subcutaneously inoculated into each mouse at a volume of 100 μL. Mice were anesthetized with 3-4% isoflurane before inoculation.

### c) Animal Grouping and Drug Administration

When tumors grew to an average size of approximately 50-80 mm3, tumor-bearing mice were randomly divided into 4 groups of 8 mice each based on body weight and tumor size. The day of group assignment and drug administration was defined as Day 0. The grouping and dosing regimen are outlined in the table below.

| | | | Dosage | Route of | |
|---|---|---|---|---|---|
| Group | The test substance | N | (mg/kg) | administration | Dosage regimen |
| 1 | Vehicle | 8 | N/A | s.c. | QD*4 weeks |
| 2 | Cisplatin | 8 | 1 | i.v. | BIW*4 weeks |
| 3 | Dimyricetin-yl-diselenid | 8 | 40 | s.c. | QD*4 weeks |
| 4 | Dimyricetin-yl-diselenid | 8 | 20 | s.c. | QD*4 weeks |

N: Number of animals per group

Dosing volume: Adjusted according to the animal's body weight at 10 uL/g

Vehicle for the test substance: 20% DMSO+60% PEG400+20% 10 mM Acetic buffer (pH4.5)

### d) Preparation of the Drug

| | | Concentration | Allocation |
|---|---|---|---|
| The test substance | Preparation Steps | (mg/mL) | frequency |
| Cisplatin | Take 1 bottle of Cisplatin (20 mg/bottle) + | 0.1 | Packaged |
| | 20 mL of sterile water for injection → 1 | | in 8 tubes |
| | mg/mL stock solution; | | Stored at −80° C. |
| | Take 1.6 mL of the stock solution, divide | | |
| | into 8 tubes, and store at −80° C. for future | | |
| | use; | | |
| | Take 1 tube of 0.2 mL stock solution each | | |
| | time and dissolve it with 1.8 mL of 0.9% | | |
| | Sodium Chloride Injection → 2 mL of G2. | | |
| Dimyricetin-yl-diselenid | Weigh 8 mg of Dimyricetin Diselenide | 4 | Allocation |
| | powder, add it to 0.4 mL of DMSO, vortex | | once a day |
| | for 1 minute; add 1.2 mL of PEG 400, vortex | | |
| | for 1 minute; add 0.4 mL of 10% HP-β-CD in | | |
| | 10 mM Acetic buffer, vortex for 1 minute and | | |
| | keep it ready for use. | | |
| Dimyricetin-yl-diselenid | Weigh 8 mg of dimyricetin diselenide | 2 | Allocation |
| | powder, add it into 0.8 mL of DMSO, vortex | | once a day |
| | for 1 minute; then add 2.4 mL of PEG 400, | | |
| | vortex for another 1 minute; add 0.8 mL of | | |
| | 10% HP-β-CD in 10 mM Acetic buffer, | | |
| | vortex for 1 minute, and set aside for future | | |
| | use. | | |

### e) Measurements

Animal Weight: Measure and record the weight of mice twice weekly after grouping.

Tumor Volume: Measure the tumor volume twice weekly after grouping for a consecutive 4 weeks. The calculation method for tumor volume (V) is as follows: V=(Length×Width²)/2. The relative tumor volume (RTV) for each nude mouse is calculated as: RTV=Vt/V0, where Vt is the measured volume for each day and V0 is the volume at the start of treatment.

Relative Tumor Proliferation Rate (T/C %): Used to evaluate the antitumor activity of the drug. The relative tumor proliferation rate T/C (%) is calculated as the mean RTV of the treatment group (T) divided by the mean RTV of the negative control group (C), multiplied by 100%.

Tumor Growth Inhibition (TG1%): TG1%=(1−T/C)×100%, where T is the mean RTV of the treatment group and C is the mean RTV of the negative control group.

Tumor Weight: At the end of the experiment, collect all tumors, weigh them, and take photos. The tumor weight inhibition rate (%) is calculated as (Mean tumor weight of the negative control group−Mean tumor weight of the treatment group)/Mean tumor weight of the negative control group×100%.

### 5) Statistical Analysis

The results will be presented as mean±S.E.M. Dunnett's multi-comparison test will be used for comparisons between two groups. A p-value <0.05 will be considered statistically significant.

### 6) Results

After drug administration, the measured tumor volumes for different mouse groups are as follows:

| | 0 day | 3 days | 5 days | 7 days | 10 days | 12 days | 14 days |
|---|---|---|---|---|---|---|---|
| TV | | | | | | | |
| (mm³) | | | | | | | |
| G1 | 75.42 | 276.77 | 371.46 | 768.24 | 1169.51 | 1689.92 | 2672.72 |
| G2 | 75.96 | 288.49 | 351.18 | 597.48 | 821.76 | 1081.49 | 1627.00 |
| G3 | 75.71 | 257.17 | 355.24 | 532.85 | 711.79 | 1066.44 | 1108.65 |
| G4 | 75.31 | 218.25 | 314.27 | 544.04 | 741.18 | 1210.83 | 1542.11 |
| TGI | | | | | | | |
| G2 | / | −3.49% | 6.13% | 22.78% | 30.23% | 36.46% | 39.56% |
| G3 | / | 9.88% | 5.58% | 34.02% | 41.86% | 38.64% | 60.23% |
| G4 | / | 29.01% | 19.28% | 32.35% | 39.14% | 29.67% | 43.53% |

The results showed that dimyricetin-yl-diselenide had a significant inhibitory effect on the liver cancer tumor model in vivo, with the 40 mg/kg dose group showing better tumor inhibition than the positive control (cisplatin). Comparing the inhibitory effects of G3 and G4 (40 mg/kg, 20 mg/kg), it was observed that the inhibition of tumors became more pronounced with the increase in the administered dose.

The above description is merely an embodiment of the present application and is not intended to limit it. For those skilled in the art, various modifications and variations of the present application are possible. Any amendments, equivalent substitutions, improvements, etc., made within the spirit and principles of the present application shall be included within the scope of the claims of the present application.

What claimed is claimed:

1. A method for dimyricetin-yl-diselenide in the preparation of a medicament for treating tumors.

2. The method according to claim 1, characterized in that the tumor is a solid tumor.

3. The method according to claim 2, characterized in that the tumor is bladder transitional cell carcinoma, breast cancer, colorectal adenocarcinoma, gastric cancer, renal cancer, liver cancer, lung cancer, melanoma, pancreatic cancer, prostate cancer, or ovarian cancer.

4. The method according to claim 3, characterized in that the liver cancer is primary liver cancer.

5. The method according to claim 1, characterized in that the tumor is hepatocellular carcinoma.

6. The method according to claim 1, characterized in that the medicament is for treating or administering to a mammal.

7. The method according to claim 6, characterized in that the mammal is a rodent or a human.

8. A method for inhibiting the growth of tumor cells in vitro in a non-therapeutic manner, characterized by including the step of adding dimyricetin-yl-diselenide to a tumor cell culture system to inhibit the growth of tumor cells.

9. The method according to claim 8, characterized in that the tumor is a solid tumor.

10. The method according to claim 8, characterized in that the tumor is bladder transitional cell carcinoma, breast cancer, colorectal adenocarcinoma, gastric cancer, renal cancer, liver cancer, lung cancer, melanoma, pancreatic cancer, prostate cancer, or ovarian cancer.
